(19) United States
(12) Patent Application Publication
(10) Pub. No.: US 2009/0105685 A1
(43) Pub. Date: Apr. 23, 2009

(54) Two Stage Jet Injection Device

(75) Inventors: Torben Stroem Hansen, Copenhagen (DK); Carsten Soerensen, Frederiksberg (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/297,960

(22) PCT Filed: Apr. 20, 2007
(86) PCT No.: PCT/EP07/53888

§ 371 Date: Nov. 19, 2008

(30) Foreign Application Priority Data
Apr. 21, 2006 (EP) 06112876.5

(51) Int. Cl. A61M 5/307

(52) U.S. Cl. 604/500; 604/70

(57) ABSTRACT

A jet injection device comprises a medical jet unit (10), a medical drug dispensing unit (20) and an medical drug injection unit (30). The empty medical jet unit (10) is connected to the medical drug dispensing unit (20) and the plunger (11) inside the medical jet unit (10) snaps to the removable drug container (40) inside the medical drug dispensing unit (20), thereby creating fluid connection between the drug container (40) and chamber of the medical jet unit (10) via a hollow back needle. The medical jet unit (10) is moved away relative to the medical drug dispensing unit (20), the plunger (11), connected to the drug container (40), moves relatively to the medical jet unit (10), which causes the impulse chamber of the medical jet unit (10) to expand and results in a dosage of liquid drug to migrate from the drug container (40) to the impulse chamber The medical jet unit (10) is then removed from the medical drug dispensing unit (20) and connected to the medical drug injection unit (30) which comprises a ram (32) and actuating means, to transmit an impulse energy to the plunger (11), whereby a dose of liquid drug is expelled.

The invention relates to a jet injection device adapted for placement against a skin surface of a subject, for injecting a dose of drug to the subject. The jet injection device integrates a dispenser unit and an injection unit into a single device.

### BACKGROUND OF THE INVENTION

Subcutaneous and intramuscular delivery of liquid drugs by injection is common in the medical arts. As some medications such as insulin must be given frequently by injection to an individual, easy performance of the injections is desirable.

Many patients dislike needle injections due to pain or fear for needles. Further, blood-borne pathogens, such as HIV and hepatitis, can be transmitted to health care workers by accidental needle-sticks. Also, the disposal of used needles is a growing concern. This disposal presents a problem to individuals other than healthcare workers. Children, for example, may find used needles in the garbage, putting them at risk of contracting infection. Discarded needles likewise pose a risk to waste disposal workers. This is at the moment a huge worldwide problem, (though partly overlooked as it mainly hits countries of low development) causing deaths counted in millions,

In efforts to minimize the fears and risks associated with needle injections, several types of needle-free jet injectors have been developed. These devices penetrate the skin using a high velocity fluid jet and deliver medication into the tissue of a patient. In order to accomplish this, a force is exerted on the liquid medication. Jet injectors in general contain a fluid drug which has been transferred into a chamber having a small orifice at one end. A drive means, e.g. a ram, is accelerated using either a coil spring or a compressed gas energy source. The ram impacts a plunger which in turn creates a high pressure impulse within the chamber. This pressure impulse ejects the fluid medicament through the orifice at high velocity, piercing the skin. The energy source continues to apply a force to the plunger which quickly propels the drug through the opening in the skin, emptying the injection chamber in a fraction of a second. The drive means may be adapted to provide a two-stage injection, i.e. a first penetrating burst of drug at a high pressure followed by a subsequent delivery of the remaining amount of drug at a lower pressure.

The energy impulse exerted on the jet injector in order to provide a sufficiently high-powered liquid jet is of a magnitude which limits the range of materials fit for withstanding this pressure. For this reason, It is at the moment not feasible to use glass to produce the injector, though glass is the preferred material to use for drug cartridges. On the other hand, plastic materials suitable for withstanding the high energy impulse during the injection has until now not been proven suitable for long term contact with drugs supposed to be injected in humans. Therefore it has been known to use a plastic material for the high strain inflicted injector part of the device, but to fill this injector only shortly prior to an injection from a glass container (cartridge) more suitable for long time storage of the drug. In this way both storing and strain issues are addressed. However it makes the steps necessary to perform a jet injection more complicated. It is known to use an adapter that connects a drug cartridge to the jet injector. Prior to injection the adapter (including cartridge) is removed uncovering the orifice. But, when the drug cartridge is an integrated part of the injection device, this principle is not feasible.

Addressing this problem, the patent document EP 1277487 discloses a cassette device for medicament reservoir for use with a jet injector system where the reservoir is isolated from the pressure waves created when the ram impact the plunger. The cassette device can use existing pre-filled cartridges and it can draw a fluid medicament directly from a multi-dose vial. The reservoir is isolated from the pressure waves by means of a tortuous pathway between the two, by means of a check valve, a flap or the like. However the device disclosed in EP 1277487 does not provide a 100% isolation between the reservoir and the jet injector and furthermore there is a risk of exposure and therefore contamination of the reservoir and the jet injector between dosages. Also the complexity of the device disclosed in EP 1277487 makes it expensive and rather complicated to operate.

US 2003/0083611 also discloses an injector which integrates a jet injector and a drug reservoir into a single device. Again there is a liquid passage between the jet injector and the drug reservoir which can be closed by a valve.

WO 2005/051465 discloses a method of filling a jet injector from a drug reservoir by means of a protruding conduit located on the proximal side of the plunger in the jet injector.

US 2002/0065483 and U.S. Pat. No. 5,190,523 shows a jet injection syringe with connection means, but without a possibility of a simple way of connecting the syringe to filling and expelling means respectively.

Other examples of known techniques of integrating a jet injector and a liquid drug reservoir are disclosed in US 2002/0007142, WO 01/89614, WO 01/37907, U.S. Pat. No. 5,480,381, U.S. Pat. No. 6,213,980, FR 2339407 and U.S. Pat. No. 5,840,062.

In view of the above, one of the objectives of the present invention is to provide a two stage jet injection device integrating a jet injection device and a drug reservoir, which is simple and of relative low cost without giving up the advantages of multi dosing and dialing a dose. Also, a main objective of the present invention is to provide a two stage jet injection device, where the connection between jet injector and drug reservoir is completely isolated and there is no exposure of neither drug nor injection orifice between injections, thereby greatly reducing the risk of contamination. Another objective of the present invention is to provide a two stage jet injection device with total blockage of the liquid passage from the jet injection device designed for dose dialing when an injection is performed, to assure an exact amount of expelled drug. Further it is a main objective of the present invention to provide a two stage jet injection device capable of using standard drug cartridges and single use jet injection nozzles.

In the alternative, it is a further objective to provide a jet injection device with resemblance of a conventional pen type injector as regards function and configuration, in order to make the patient comfortable with the jet injection device and so that the jet injection device can easily be utilized by a non-professional user, e.g. a insulin requiring diabetic.

### SUMMARY OF THE INVENTION

In the disclosure of the present invention, embodiments and aspects will be described which will address one or more of the above objectives or which will address objectives apparent from the below disclosure as well as from the description of exemplary embodiments.

In a first aspect, a two stage jet injection device is provided comprising a built together dispenser unit containing a drug cartridge and a medical drug injection unit. The dispenser unit has means to fix a drug reservoir, such as a standard drug cartridge inside the hollow body of the dispenser unit. In this way, when fixed to the dispenser, the cartridge will follow the movement of the dispenser. The cartridge can be made of any suitable and approved material for storing of drugs.

Inside the medical drug injection unit, there are actuation means to provide the actuation of the plunger of the jet-injector when a dose is being expelled from the jet-injector. The actuation means will not be described further, as they are not the issue of the present invention, but they can be any of known actuators driven by chemical combustibles, compressed gas, one or more mechanical springs, magnetic force etc. As mentioned, the drive means may be adapted to provide a two-stage injection, i.e. a first penetrating burst of drug at a high pressure followed by a subsequent delivery of the remaining amount of drug at a lower pressure. By assembling the medical drug dispensing unit and the medical drug injection unit into one single device, ease of use is provided to the patient both during and between injections, as few parts are involved.

Both the dispensing-and the medical drug injection unit are adapted to releasable connect to a medical jet unit. In one embodiment, the medical jet unit is equipped with a thread which fits into a corresponding thread on the dispensing-and the medical drug injection unit. When the medical jet unit is screwed on to the medical drug dispensing unit via the thread, a protruding conduit such as a back needle mounted on the back side of the plunger of the medical jet unit will penetrate the rubber closure on the drug cartridge, thereby making a fluid passage between the drug reservoir and the impulse chamber in the medical jet unit. The back needle can be made of any suitable material such as steel or plastic or fiber enforced materials. When fully screwed onto the medical drug dispensing unit, gripping means also positioned on the back side of the plunger will automatically hook on to the neck of the cartridge forced by the relative axial movement between the medical jet unit and the dispensing unit via the thread, thereby making a snap connection between the plunger and the cartridge. To dial a dose, the medical jet unit is then screwed off the medical drug dispensing unit again. As the cartridge is fixed to the medical drug dispensing unit, and the plunger is snapped onto the cartridge, moving the medical jet unit away from the medical drug dispensing unit will move the plunger relative to the housing of the medical jet unit. This causes for the impulse chamber bordered by the housing and the plunger to expand. The orifice of the medical jet unit is sealed off and blocked by a closure integrity member, and the space between the medical jet unit housing and the plunger is likewise sealed, therefore the expansion of the impulse chamber creates an under-pressure in the impulse chamber relative to the surroundings, which causes fluid drug to migrate from the cartridge to the impulse chamber via the fluid connection. The migration continues until the plunger hits a defined stop in the medical jet unit housing. Further unscrewing of the medical jet unit then causes the snap lock to automatically open, as the plunger can no longer move further relative to the housing. As the snap lock opens, the further unscrewing of the medical jet unit from the medical drug dispensing unit causes the back needle to be withdrawn from the cartridge septum, thereby sealing of the drug in the cartridge to the surroundings.

A dose is now dialed into the medical jet unit. To expel this dose, the medical jet unit has to be connected to the medical drug injection unit. To do this, the medical jet unit is screwed onto the medical drug injection unit, which (like the medical drug dispensing unit) is equipped with a thread, corresponding the thread on the medical jet unit. As the back needle forms a fluid passage from the surroundings to the impulse chamber containing the fluid drug, this fluid passage needs to be blocked prior to an injection in order to avoid backflow of the fluid. This is done via a plug mounted on the ram on the medical drug injection unit. When screwing on the medical jet unit to the injection device, the back needle penetrates into the massive plug, thereby blocking the fluid passage in the back needle. When the medical jet unit is fully on-screwed, the ram of the injection device rests against the plunger of the medical jet unit, and the injection device is armed and ready for expelling the drug by releasing the drive means in the medical drug injection unit.

The assembled built together device can in one embodiment have the shape of two parallel assembled pen device, but can also appear in a range of other shapes like box-shape, a single elongated shape with dispensing-and medical drug injection unit in either, this list by no means being exhaustive. Likewise, the connection between on one hand the medical jet unit, and on the other hand the dispensing-and the medical drug injection unit can be in a range of other types than the aforementioned thread connection. Obvious alternatives are a bayonet connection, a ball and socket connection and hinged and locked connections.

In a further embodiment, the built-together pen device can have a cover either loose or hinged, to cover the mounting openings of the medical drug dispensing unit and the medical drug injection unit, to minimize the exposure to the surroundings.

As the two stage jet injection device is suitable for single-use medical jet units, which can be stored inside a sterilized enclosure, it can also be an advantage to equip the injection device with a temporary storage for one or more medical jet units. This storage can be inside the device, or it can be a part of the container or bag designed to keep the injection device.

In a further embodiment, the invention provides a jet expelling device of the above-described type, further comprising a dose setter for selectable setting a dose of drug to be expelled, an actuator for actuating the impulse generating system and the drive assembly, and an actuatable release, wherein actuation of the release causes the impulse generating system to expel a portion of the set dose from the impulse chamber at a high pressure through the outlet nozzle, followed by subsequent expelling of the remaining portion of the set dose from the impulse chamber through the outlet nozzle by means of the drive assembly. The dose setting can in one embodiment be made by varying the distance between the stop and the distal end of the medical jet unit, the dialing distance, instead of having a well defined stop. The dialing distance can be varied by means of a second thread connection, indents or distance rings on the medical jet unit.

### DESCRIPTION OF THE DRAWINGS

In the following the invention will be further described with references to the drawings, wherein

FIG. 1 shows a well known principle of connecting a medical jet unit to a drug cartridge via an adapter in sectional view.

FIG. 2 shows the sequences of dispensing and expelling via the two stage jet injection device in perspective view.

FIG. 3 shows the steps of the dispensing sequence in perspective view.

FIG. 4 shows the steps of the expelling sequence in perspective view.

In the figures like structures are generally identified by like reference numerals.

### DESCRIPTION OF EXEMPLARY EMBODIMENTS

When in the following terms as “distal”, “proximal” and “radial” or similar relative expressions are used, these only refer to the appended figures and not necessarily to an actual situation of use. The shown figures are schematic representations for which reason the configuration of the different structures as well as there relative dimensions are intended to serve illustrative purposes only.

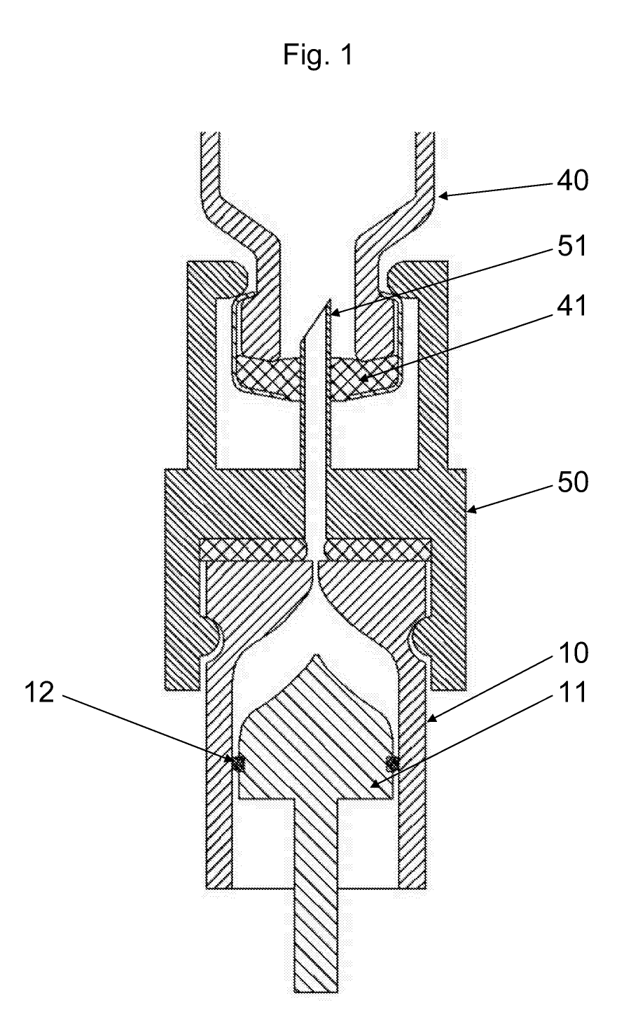

FIG. 1 shows a well known principle of the connection of a medical jet unit 10 to a drug cartridge 40 by means of an adapter 50. An impulse chamber is defined between the housing of the medical jet unit 10, the plunger 11 and the sealing 12. The adapter 50 is equipped with a needle 51, which can penetrate the septum 41 of the cartridge 40 to establish fluid connection between the impulse chamber and the cartridge 40 interior when the adapter 50 is connected to the medical jet unit 10 and the cartridge 40 both.

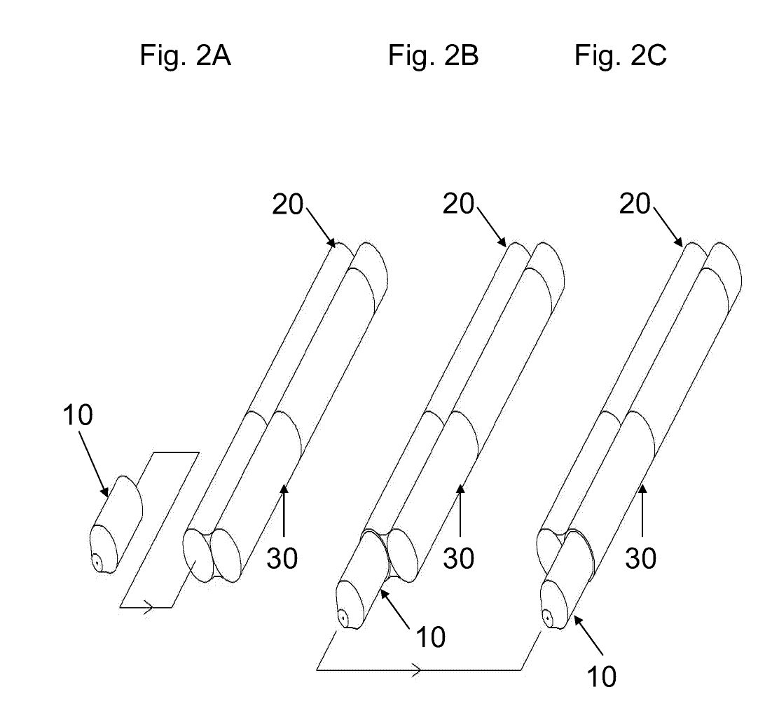

FIG. 2 shows the sequences of dispensing and expelling a liquid drug from a two stage medical jet unit device according to the invention. A combined medical drug dispenser unit 20 and medical drug injector unit 30 is in turn connected to a medical jet unit 10. First the medical jet unit 10 is connected to the medical drug dispensing unit 20 where after a dose is dialed whereby a predetermined amount of liquid drug corresponding to one injection is migrated from the cartridge contained in the dispenser unit 20 to the impulse chamber of the medical jet unit 10. Next the medical jet unit 10 is removed from the dispenser unit 20 and connected to the injector unit 30 and the two stage jet injection device is ready to inject a dose through the skin of a subject.

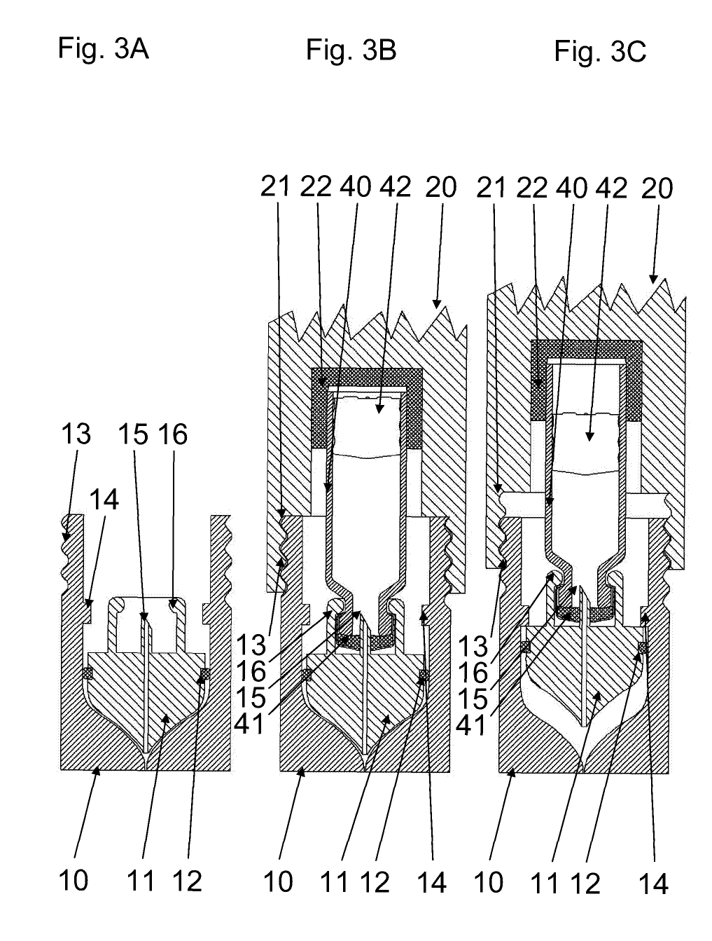

FIG. 3 shows in greater detail the steps of the dispensing sequence. FIG. 3A shows a medical jet unit 10 comprises a housing, a plunger 11 with a protruding conduit such as a back needle 15 in fluid connection with the impulse chamber defined between the inside housing 10, the plunger 11 and the sealing 12. The plunger 11 as well as the housing 10 are equipped with connection means 16/13, in this embodiment a snap connection 16 and a thread 13. Further the housing 10 interior comprises a stop 14 defining a distance of free movement of the plunger 11 inside the housing 10. On FIG. 3B a medical drug dispensing unit 20 is shown, comprising fixation means 22, holding a cartridge 40 containing a fluid drug contained by the cartridge walls 40, the cartridge plunger 42 and the septum 41. The medical jet unit 10 is connected to the medical drug dispensing unit 20, in this embodiment by screwing the two units together by means of the thread connection 21/13. When the medical jet unit 10 and the dispenser unit 20 is completely assembled, the snap connection 16 has embraced the neck of the cartridge 40 and the back needle 15 has penetrated the septum 41, thereby establishing fluid connection between the liquid drug contained in the cartridge 40 and the impulse chamber of the medical jet unit 10. When next the medical jet unit 10 is un-screwed from the dispenser unit 20, the snap connection 16 causes the plunger 11 to stay fixed in relation to the cartridge 40, which is again fixed to the dispenser unit 20. The plunger 11 therefore moves in proportion to the medical jet unit housing 10 whereby an under pressure is created in the impulse chamber. This causes liquid drug to migrate from the cartridge 40 to the impulse chamber until the plunger 11 hits the stop 14. To achieve this, it is important that the connection 22 between the cartridge 40 and the dispenser unit 20 is not air tight, to ensure that the cartridge plunger 42 can move freely without an up-build of under pressure in the space between the connection 22 and the cartridge plunger 42. When further the medical jet unit 10 is totally un-screwed from the dispenser unit 20, the stop 14 causes the plunger 11 to move in relation to the dispenser unit 20, whereby the snap connection 16 is forced to again release the grip around the cartridge 40 neck. The dispenser unit 20 and the medical jet unit 10 is then disconnected, and the medical jet unit 10 is loaded with an amount of liquid drug contained in the impulse chamber, corresponding a single dose of drug.

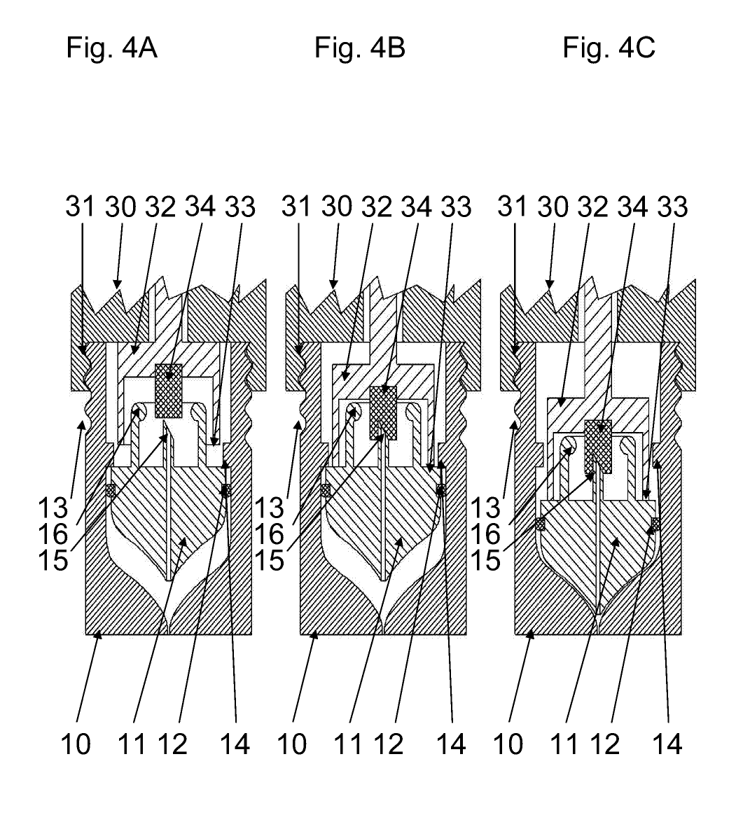

FIG. 4 shows in greater detail the steps of the expelling sequence. On FIG. 4A the medical jet unit 10 has been mounted on the injector unit 20 in this embodiment by means of the thread connection 13/21. As can be seen, the injector unit 20 comprises a ram 23 which has a distance for free movement before the front edge 24 of the ram contacts the plunger 11 when expelling a drug. This distance serves to ensure a high energy impulse during the first stage of the injection. However, when turning to FIG. 4B, it is shown how this free distance can be exploited also to block the back needle 15 by means of a plug 25 mounted on the ram 23, to avoid any back flow of liquid drug during an injection. As the whole liquid passage through the back needle 15 to the impulse chamber is liquid filled from the dispensing sequence, the media in the liquid passage is incompressible and there will be no flex or elasticity which could soften the jet impulse. Because there is a considerable distance along the liquid passage from the back needle 15 to the impulse chamber, no contamination can reach the drug which is injected during only fractions of a second. However, as the combined dispensing-and injecting unit is a durable device, the connection between the ram 23 and the plug 25 allows for replacement of the plug 25, which also secures a fluid tight blockage of the back needle 15. After blocking of the back needle 15 during the initial step of the expelling, the front end 24 of the ram 23 reaches the plunger 11 and an impulse is delivered to the plunger 11 and further to the liquid in the impulse chamber, which is then expelled under high pressure/speed at least during the initial stage of the injection. On FIG. 4C the plunger 11 has reached the bottom of the impulse chamber and the total amount of liquid has been expelled. As the injection is now complete, the used medical jet unit 10 can again be unscrewed and removed from the medical drug injection unit 20.

1. A medical device system comprising:
a medical jet unit comprising a housing with first connecting means and a cavity defining an impulse chamber, which cavity comprises a distal opening and a proximal opening, wherein the distal opening has the form of an orifice, and the proximal opening is sealed by a slideable plunger, which plunger comprises a distal side related to the cavity and an opposite proximal side wherein the plunger further comprises a fluid passage creating liquid communication between the cavity and the proximal side of the plunger, which proximal side is further provided with a protruding conduit connected to the fluid passage, said plunger is provided with second connecting means at the proximal side,
a medical drug dispenser unit for filling said medical jet unit with a dose of liquid drug, the medical drug dispenser unit comprising a drug reservoir with a distal and a proximal end, wherein said medical drug dispenser unit comprises first connecting means connecting to the first connecting means of the jet unit, and second connecting means on the distal end of the drug reservoir connecting to the second connecting means of the medical jet unit,
and a medical drug injection unit for forcing the expelling of a dose of liquid drug from said medical jet unit, the medical drug injection unit comprising a ram adapted to be driven towards the plunger, thereby creating an impulse on said plunger, having first connecting means connecting to the first connecting means of the medical jet unit, and means for sealingly engaging said protruding conduit,
wherein the first connecting means of the medical jet unit are adapted to engage to the first connecting means of the medical drug dispenser unit or the drug injection unit respectively, and that the second connecting means of the medical jet unit are adapted to automatically engage or disengage to the second connecting means of the drug reservoir when the medical jet unit is engaged or disengaged to the medical drug dispenser unit via the first connecting means.

2. A medical device system according to claim 1, wherein the medical drug injection unit comprises sealing means for sealingly engaging said protruding conduit.

3. A medical device system according to claim 2, wherein the protruding conduit is automatically sealingly engaged by said sealing means when the medical jet unit is engaged to the medical drug injection unit via the first connecting means.

4. A medical device system according to claim 1, wherein the medical jet unit further comprises stop means defining a limit to the sliding movement of said plunger.

5. A medical device system according to claim 4, wherein the distance from said stop means to the distal end of said medical jet unit is variable, thereby enabling a variable dose to be dialed and dispensed from the medical drug dispenser unit to the medical jet unit.

6. A medical device system according to claim 1, wherein the medical drug dispenser unit and the medical drug injection unit are build together into a single integrated two stage jet injection device.

7. A medical device system according to claim 1, wherein the first connecting means is a thread connection, a bayonet connection, a snap connection or a ball and ring connection.

8. A medical device system according to claim 1, wherein the medical jet unit is a disposable unit comprising a closure integrity member to seal and block the orifice prior to an injection.

9. A medical device system according to claim 1, wherein the medical jet unit can contain a variety of standard injection volumes.

10. A medical device system according to claim 1, wherein said drug reservoir comprises a pierceable membrane at the distal end for engagement of said protruding conduit, thereby establishing fluid connection between the drug reservoir and said fluid passage of the plunger.

11. A medical device system according to claim 1, wherein the medical drug dispenser unit further comprises a spring which is compressed when the housing is connected to the medical drug dispenser unit.

12. A medical device system according to claim 1, wherein the drug reservoir is a replaceable cartridge comprising a sealing membrane and a slideable cartridge plunger.

13. A medical device system according to claim 1, wherein said sealing means is a plug which engages the protruding conduit thereby blocking the fluid passage.

14. A method for dispensing a liquid drug and arming a medical drug injection unit before expelling the liquid drug, comprising:
connecting a medical jet unit to a medical drug dispensing unit via first connection means, and simultaneously connecting a slideable plunger integrated in said medical jet unit to a drug reservoir inserted and fixed in said medical drug dispensing unit via second connection means, whereby a protruding conduit of said plunger engages a membrane of said drug reservoir, thereby creating a fluid passage between said drug reservoir and an impulse chamber in the medical jet unit,
initiating disconnection of said medical jet unit from said medical drug dispensing unit, which in the initial step of the disconnecting faze causes the medical jet unit housing to move away from the medical drug dispensing unit while the plunger remains connected to the drug reservoir, this relative movement of the plunger in relation to the medical jet unit housing causing the volume of the impulse chamber to expand and the resulting under-pressure in the impulse chamber relative to the surroundings causes a dose of liquid drug to migrate from the drug reservoir to the impulse chamber,
further disconnecting the medical jet unit from the medical drug dispensing unit, whereby the plunger hits stop means comprised in the housing to limit the movement of the plunger relative to the housing, causing the plunger to be released from the drug reservoir, and connecting the medical jet unit to a medical drug injection unit comprising a ram and actuating means to provide an impulse energy to the plunger.

15. A medical device system according to claim 9, wherein the variety of standard injection volumes comprises volumes of 100, 200 and 300 micro litres.
